(19) United States
(12) Patent Application Publication
(10) Pub. No.: US 2022/0125476 A1
(43) Pub. Date: Apr. 28, 2022

(54) Surgical Needle

(71) Applicants: TEXAS HEART INSTITUTE, Houston, TX (US); BAYOR COLLEGE OF MEDECINE, Houston, TX (US)

(72) Inventors: ANAND GANAPATHY, Los Angeles, CA (US); DAVID BURKLAND, Houston, TX (US); ALLISON POST, Houston, TX (US); BRIAN GREET, Houston, TX (US); MEHDI RAZAVI, Houston, TX (US); MATHEWS JOHN, Houston, TX (US)

(21) Appl. No.: 17/427,229

(22) PCT Filed: Feb. 6, 2020
(86) PCT No.: PCT/US2020/016976

§ 371 Date: Jul. 30, 2021

Related U.S. Application Data
(60) Provisional application No. 62/802,559, filed on Feb. 7, 2019.

(51) Int. Cl. A61B 17/34; A61B 34/20

(52) U.S. Cl. CPC A61B 17/3403; A61B 2017/00026; A61B 34/20

(57) ABSTRACT

A surgical needle is configured to detect whether a distal tip of the surgical needle perforates or ends up in an undesirable location, or detect whether the distal tip of the surgical needle has accessed a desired location. The surgical needle includes a hub and a body connected to the hub. The body has a hollow core and includes a sharp-pointed tip at a distal end. A first electrode is formed by the sharp-pointed tip of the body. At least a second electrode is provided around the body. The first and second electrodes are connected to a wired connector that can be plugged into an external sensing system. The external sensing system can monitor impedance, electrical parameters, or other parameters.

### BACKGROUND

This disclosure relates generally to surgical needles. This disclosure relates more particularly to surgical needles that are configured to detect whether a distal tip of the surgical needle perforates or ends up in an undesirable location, and/or detect whether the distal tip of the surgical needle has accessed a desired location.

Medical devices used typically to gain surgical access include, for example, needles, trocars, veress needles, biopsy needles, etc. These devices have a distal needle-like tip, a cannula, and a proximal hub. Surgical access using these devices is sometimes complemented by the use of imaging techniques such as ultrasound and fluoroscopy.

Surgical access can present complications wherein the distal tip of these medical devices perforates or ends up in an undesirable location. These complications may include potential pneumothorax/hemothorax during vascular or pericardial access procedures, liver perforations during pericardial access procedures, and puncturing large blood vessels or abdominal organs during trocar laparoscopic surgeries.

Application pub. no. WO 2018/175348 discloses a surgical instrument that includes an impedance sensing system for monitoring the position of the tip of the surgical instrument relative to the pericardial space. The surgical instrument can consist of a guidewire or needle including a conductive core terminating at a distal tip of the guidewire or needle. The distal tip has a conductive surface exposed to patient tissues and/or fluids. The impedance sensing system includes a first electrode formed by the exposed surface of the guidewire or needle, and at least a second electrode isolated from the conductive core of the guidewire or needle. The second electrode may be formed on an outer surface of the guidewire or needle, or may be a pad electrode applied to the patient skin. The impedance sensing system also includes an impedance analyzer for measuring impedance and phase using one or more frequencies. Having a first electrode formed by the exposed surface of the guidewire or needle allows a more accurate measurement of the impedance of the tissues in front of the needle. However, the insulation of such a first electrode from the rest of the surgical instrument can be difficult.

Therefore, there is a continuing need for a surgical needle that can be used to detect pneumothorax, hemothorax, puncture of the liver/spleen/kidney, injury to hollow organs (including stomach, colon, and small bowel), bleeding complications and other complications related to surgical access. Preferably, the surgical needle can also be used to confirm access to the desired location during surgery. For example, the surgical needle could confirm vascular access during cardiovascular procedures such as cannulation of the femoral and jugular vessels, or pericardial access.

### BRIEF SUMMARY OF THE DISCLOSURE

The disclosure describes a surgical needle that is configured to detect whether a distal tip of the surgical needle perforates or ends up in an undesirable location, and/or detect whether the distal tip of the surgical needle has accessed a desired location.

The surgical needle may comprise a hub and a body connected to the hub. The body may have a hollow core. The body may include a sharp-pointed tip at a distal end. The body may comprise a sharp-pointed extension attached to a tube. A first electrode may be formed by the sharp-pointed tip of the body. At least a second electrode may further be provided around the body.

In some embodiments, the body may be made of conductive material, such as metal. An outer coating may be provided on a portion of an outer surface of the body. The outer coating may be made of a non-conductive material. The outer coating may not be provided on the first electrode. The at least second electrode may be provided around the outer coating. The surgical needle may further comprise an inner coating provided on at least a portion of an inner surface of the body inside the hollow core of the body.

In some embodiments, the sharp-pointed tip may be formed by a sharp-pointed extension and a first tube that is to the sharp-pointed extension. The first tube may be made of a non-conductive material. The sharp-pointed extension may be made of a conductive material. The at least second electrode may be provided around the tube. The first tube is connected to the hub or the surgical needle may further comprise a second tube connected between the first tube and the hub. The second tube may be of a conductive material or of a non-conductive material.

### BRIEF DESCRIPTION OF THE DRAWINGS

For a more detailed description of the embodiments of the disclosure, reference will now be made to the accompanying drawings, wherein:

FIG. 1 is a side view of an embodiment of a surgical needle;

FIG. 2 is a sectional view of a body of the surgical needle shown in FIG. 1;

FIG. 3 is a side view of another embodiment of a surgical needle; and

FIG. 4 is a side view of yet another embodiment of a surgical needle.

### DETAILED DESCRIPTION

The disclosure describes exemplary designs of a surgical needle. In some embodiments, the surgical needle may be used to access tissue such as muscle including the myocardium, the pericardium, lymph nodes, solid/viscus organs etc., a potential tissue-space such as a peritoneum, retroperitoneal sac, dural sac, etc., hollow structures such as the vasculature, abscesses, or viscus organs, and other named structures such as nerves, muscles, etc., and even abnormal tissue such as cancerous, ischemic, necrotic tissue.

In some embodiments, a wired connector may connect electrodes of the surgical needle to a sensing system that can monitor electrical activity of the heart, electrical conduction of the muscles/nerves, analyze impedance, or measure properties such as oxygen saturation/content, pH, osmolality, glucose, tissue health, composition, etc., or a combination thereof. The sensing system may include a user interface to display electrical activity or other measurement.

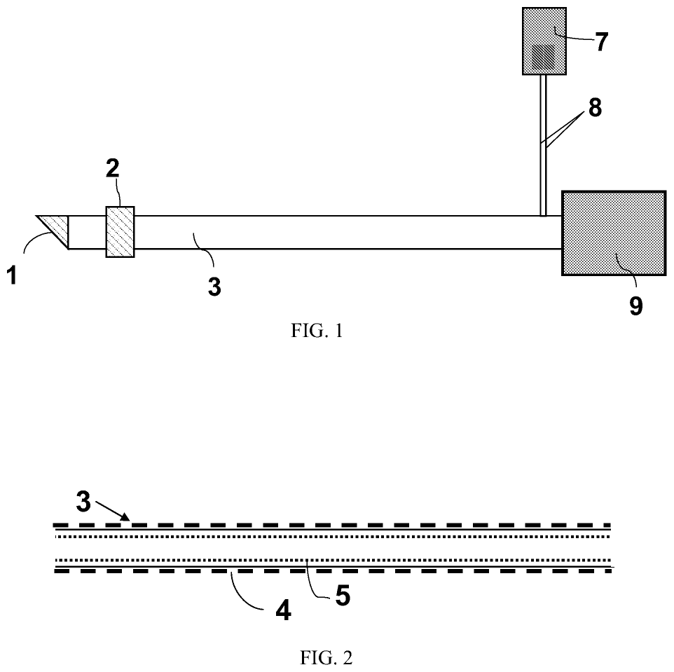

FIG. 1 illustrates an example design of a needle. The needle includes a body 3 that may be unitary. The body 3 has a sharp-pointed tip at its distal end and is made of conductive material (such as a metal). The body 3 has non-conductive material on a portion of the outer surface (such as outer coating 4 shown in FIG. 2). The body 3 may optionally have non-conductive material on the inner surface (such as inner coating 5 shown in FIG. 2). The needle also includes a hub 9. The hub 9 may be made of a conductive or non-conductive material and will be used by a practitioner for precisely handling the needle as it is advanced and/or retracted in a patient's body.

Two electrodes may be used for measuring impedance. Electrode 1 comprises the sharp-pointed tip of the needle, and thus is made of a conductive material (such as a metal). In the example design of FIG. 1, electrode 1 is thus integral to the body 3. Electrode 1 does not have non-conductive material (e.g., coating) on the outer surface. Electrode 2 comprises a ring or a band of conductive material wrapped preferably around the entire exterior of the body 3 of the needle. The two electrodes 1 and 2 are connected to wires 7 and connector 8 that can form a wired connector. The wired connector can be plugged into an external sensing system that can monitor impedance, electrical parameters, or other parameters.

The body 3 may be hollow to allow for the passage of devices and substance.

In other embodiments, the body 3 of the needle may be segmented. Accordingly, the body 3 may be formed by two portions: a sharp-pointed extension attached to a tube. The sharp-pointed extension may serve as electrode 1, and is thus made of conductive material. The sharp-pointed extension does not have non-conductive material on the outer surface, and may or may not have non-conductive material on the inner surface.

FIG. 2 illustrates a portion of the body 3 of the needle shown in FIG. 1, in a sectional view. The body 3 of the needle may be hollow to allow for other devices or substances to pass through. The dashed lines represent a non-conductive outer coating 4 provided around the body 3 of the needle. This coating electrically insulates the electrode 2 shown in FIG. 1 from the body 3 of the needle. There is also a non-conductive coating 5 provided on the inner surface of the body 3 shown by dotted lines.

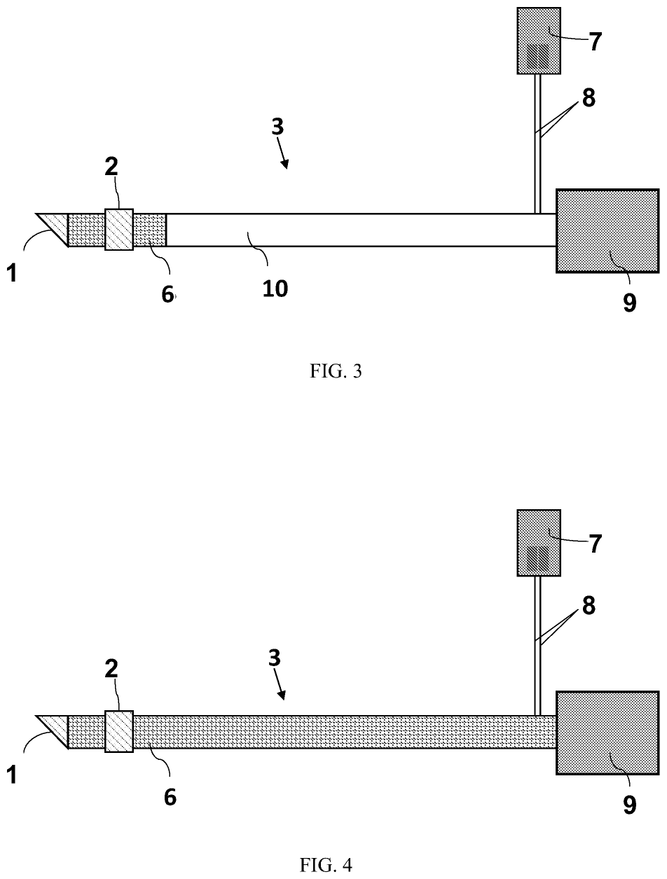

FIG. 3 illustrates another exemplary design of a needle. The body 3 of the needle is composite. The body 3 of the needle can include a non-conductive material that connects to and provides electric insulation from electrode 1, for example, formed by an entirety of the first tube 6, which would be made of a non-conductive material. Accordingly, the body 3 is formed by three portions that are attached together: a sharp-pointed extension forming electrode 1, a first tube 6 attached to the sharp-pointed extension, and a second tube 10 attached to the first tube 6. The first tube 6 does not extend to the hub 9, but the second tube may extend to the hub 9. Thus, the non-conductive tube 6 may form a middle portion of the needle and the first tube 6 provides a mechanical connection between the sharp-pointed extension forming electrode 1 and the second tube 10 forming a remaining portion of the body 3. The second tube 10 may be made of the same material as electrode 1 or may be an extension of the first tube 6.

The sharp-pointed extension serves as a needle tip. The sharp-pointed extension comprises a conductive material. In some embodiments, an entirety of the conductive sharp-pointed extension can also serve as a distal tip electrode 1. Electrode 2 is preferably provided on the first tube 6. As such, the first tube 6, which is non-conductive, can act as an electric insulator for the distal tip electrode 1 and any electrode, such as electrode 2, provided along the length of the first tube 6.

The electrode 1 formed by the sharp-pointed extension, the first tube 6, and the second tube 10 may be hollow to allow for the passage of devices and substance.

The two electrodes 1 and 2 are connected to a wired connector that can be plugged into an external sensing system. The wired connector includes wires 7 and connector 8. The external sensing system can monitor impedance, electrical parameters, or other parameters.

FIG. 4 illustrates yet another exemplary design of a needle. The body 3 of the needle is formed by two portions that are attached together: a sharp-pointed extension that is forming electrode 1, and a tube 6 attached to the sharp-pointed extension. The sharp-pointed extension serves as the needle tip. The first tube 6 extends to the hub 9. The tube 6 provides a mechanical connection between the sharp-pointed extension and the hub 9. The tube 6 is preferably made of a non-conductive material such that the tube 6 provides electrical insulation between electrode 1 and hub 9. The sharp-pointed extension comprises a conductive material so that its entirety can optionally serve as electrode 1. The tube 6 may be surrounded by one or more electrodes distributed along its length, such as electrode 2.

The electrode 1 formed by the sharp-pointed extension, and tube 6 may be hollow to allow for the passage of devices and substances.

The two electrodes 1 and 2 are connected to a wired connector (including wires 7 and connector 8) that can be plugged into an external sensing system. The external sensing system can monitor impedance, electrical parameters, or other parameters.

Any or all of the embodiments disclosed in FIGS. 1-4 may be modified in any or all of the following ways. The wired connector (including wires 7 and connector 8) to the sensing system may be at least partially replaced by a wireless connection. The two electrodes 1 and 2 may be replaced by three or more electrodes. Accordingly, bipolar impedance measurements may be replaced by multipolar impedance measurements. Impedance measurements may include impedance magnitude, impedance phase, or both impedance magnitude and phase. The external sensing system can further include algorithms configured to calculate a position of the device relative to, for example, an organ, an organ space from measurements of impedance magnitude, impedance phase, or both impedance magnitude and phase. For example, a bipolar impedance measurement performed by a pair of electrodes, one of which is located at the needle tip could identify when a needle passes through the fascia and into the ascites filled peritoneal cavity. The sensing system may further be connected to additional electrodes for the measurement of impedance magnitude and/or phase in locations of a patient's body that are remote from the needle. As such, the impedance values measured with the electrodes provided on the needle can be normalized, such as normalized by an impedance characteristic of the inferior vena cava or an impedance characteristic of the aorta. Thus additional electrodes connected to the sensing system may be inserted into the inferior vena cava or the aorta and used for real-time normalization of the impedance values measured with the electrodes provided on the needle. Conversely, the impedance values measured with the electrodes provided on the needle may be used for real-time normalization of the impedance values measured with a device inserted into the inferior vena cava or the aorta.

In view of the foregoing, a person having ordinary skill in the art, given the benefit of this disclosure, can appreciate that the surgical needles described herein can be used by practitioners in procedures where a commonly used needle is currently used, for example, where a 21G micropuncture needle is used for accessing the pericardial sac through the subxiphoid process. Compared to a commonly used needle, the surgical needles described herein may provide additional information to the practitioner through impedance measurements, preferably performed continuously and/or in real-time, among other parameters. Thus, the surgical needles described herein may be connected to an external sensing system, including an impedance analyzer, which in turn may be connected to a monitor with a user interface. The impedance data can be used to determine the location of the tip of the needle, instead of, or in addition to, fluoroscopic guidance. In some applications, the surgical needles described herein can be used to access tissues, and determine the position of the tip of the needles relative to tissues such as muscle including the myocardium, the pericardium, lymph nodes, solid/viscus organs etc., a potential tissue-space such as a peritoneum, retroperitoneal sac, dural sac, etc., hollow structures such as the vasculature, abscesses, or viscus organs, and other named structures such as nerves, muscles, etc., and even abnormal tissue such as cancerous, ischemic, necrotic tissue. In particular, the surgical needles described herein can be used to detect pneumothorax, hemothorax, puncture of the liver/spleen/kidney, injury to hollow organs (including stomach, colon, and small bowel), bleeding complications and other complications related to pericardial access. Furthermore, the surgical needles described herein can be used to confirm access to the desired location during surgical access. For example, the surgical needles described herein could be used to confirm vascular access during cardiovascular procedures such as cannulation of the femoral and jugular vessels. The electrodes provided on the surgical needles described herein can further be used to characterize the composition of a tissue it has entered. In some embodiments, these electrodes could be used to measure tissue compositions, for example, the percentage of fat versus tissue. In some embodiments, these electrodes may be used to assess the severity of a disease or complication. For example, in some embodiments, in the event of a pericardial effusion during pericardial access, the surgical needles described herein may be able to assess the amount of effusion in the pericardial sac.

The invention is susceptible to various modifications and alternative forms. Specific embodiments of the invention are shown by way of example in the drawings and description. It should be understood, however, that the drawings and detailed description thereto are not intended to limit the claims to the particular form disclosed, but on the contrary, the intention is to cover all modifications, equivalents, and alternatives falling within the scope of the claims.

What is claimed is:

1. A surgical needle comprising:
a hub;
a body connected to the hub, wherein the body has a hollow core, wherein the body is made of conductive material, wherein the body includes a sharp-pointed tip at a distal end;
an outer coating provided on a portion of an outer surface of the body, wherein the outer coating is non-conductive;
a first electrode formed by the sharp-pointed tip of the body, wherein the outer coating is not provided on the first electrode; and
at least a second electrode provided around the outer coating.

2. The surgical needle of claim 1 further comprising an inner coating provided on at least a portion of an inner surface of the body inside the hollow core of the body.

3. The surgical needle of claim 1 or 2, wherein the body comprises a sharp-pointed extension attached to a tube.

4. A surgical needle comprising:
a hub;
a body connected to the hub, wherein the body has a hollow core, wherein the body includes a sharp-pointed extension that is made of conductive material, wherein the body includes a first tube made of a non-conductive material that is attached to the sharp-pointed extension;
a first electrode formed by the sharp-pointed extension; and
at least a second electrode provided around the tube.

5. The surgical needle of claim 4 wherein the first tube is connected to the hub.

6. The surgical needle of claim 4 further comprising a second tube connected between the first tube and the hub.

7. The surgical needle of claim 6 wherein the second tube is made of a conductive material.

8. The surgical needle of claim 6 wherein the second tube is made of a non-conductive material.
